(19) United States
(12) Patent Application Publication
(10) Pub. No.: US 2024/0374418 A1
(43) Pub. Date: Nov. 14, 2024

(54) CREATING A DRUG DISPENSING OPHTHALMIC DEVICE WITH INTEGRATED MODULAR ELEMENTS

(71) Applicant: TWENTY TWENTY THERAPEUTICS LLC, South San Franciso, CA (US)

(72) Inventor: Christian GUTIERREZ, South San Francisco, CA (US)

(21) Appl. No.: 18/195,866

(22) Filed: May 10, 2023

(51) Int. Cl. A61F 9/00; G02C 7/04; G02C 11/00

(52) U.S. Cl. CPC A61F 9/0017; G02C 7/04; G02C 11/10

(57) ABSTRACT

A drug dispensing ophthalmic device can be constructed from integrated modular elements. First, an integrated device can be created by mating an interconnect interface of an electronic control module (ECM) to a passive interconnect interface of a primary drug container module (PDCM) and forming an electrical connection between the interconnect interface of the ECM to the passive interconnect interface of the PDCM. Then, the integrated device can be encapsulated within a body of an ophthalmic device.

### TECHNICAL FIELD

The present disclosure relates to a drug dispensing ophthalmic device, and, more specifically, to systems and methods for creating the drug dispensing ophthalmic device with integrated modular elements.

### BACKGROUND

Active drug dispensing ophthalmic devices employ electronic control components to facilitate drug delivery to a patient's eye. These drug dispensing ophthalmic devices can release a customized dose of one or more drugs at one or more programmable times. However, the integration of drugs and electronic components in a single device for ophthalmic use can be difficult, expensive, and unduly limiting. Challenges arise with drug dispensing ophthalmic devices due to differences between packaging requirements, sterilization requirements, storage instructions, and shelf life of the one or more drugs and electronic components. For example, the requirements for storing and sterilizing the one or more drugs are often counter to or even unnecessary for different drugs and/or for any active electronic components. Additionally, shelf life of the entire ophthalmic device is based on only the shortest shelf life of the components (often the one or more drugs).

### SUMMARY

Described herein are systems and methods for creating a drug dispensing ophthalmic device with integrated modular elements. The modular elements can include separate electronics containing modules and drug containing modules, allowing both modules to be packaged, sterilized, and stored independently. The electronics containing module (ECM) can be active, while the one or more primary drug containing modules (PDCM) can be passive and receive an electrical signal from the active electronics containing module.

In one aspect, the present disclosure includes a method for creating the drug dispensing ophthalmic device with integrated modular elements. An integrated device can be created by mating an interconnect interface of an ECM to a passive interconnect interface of a PDCM; and forming an electrical connection between the interconnect interface of the ECM to the passive interconnect interface of the PDCM. The integrated device can be encapsulated within a body of an ophthalmic device.

In another aspect, the present disclosure includes the ophthalmic device that can be formed with different modules. An ECM can include an interconnect interface. A PDCM can include a passive interconnect interface. The passive interconnect interface of the PDCM can mated with the interconnect interface of the ECM. A body made of a biocompatible material safe for ocular wear can encapsulate the mated modules.

In a further aspect, the present disclosure includes a method for creating the drug dispensing ophthalmic device with two or more drugs to be delivered using integrated modular elements. A first PDCM for a patient can be chosen based on a disorder of an eye of the patient and at least one drug stored in the first PDCM. At least a second PDCM for the patient can be chosen based on the disorder of the eye of the patient and at least one other drug stored in the second PDCM. The first PDCM can be mated to a first interconnect interface of an ECM to form an integrated device and at least the second PDCM can be mated at least a second interconnect interface of the ECM to add to the integrated device. The integrated device can be encapsulated within in a body of an ophthalmic device.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is an illustration showing creation of a drug dispensing ophthalmic device with integrated modular elements;

FIG. 2 shows illustrations of examples of fabrication of interconnect interfaces of the integrated modular elements of FIG. 1;

FIG. 3 is a diagram showing an example electronics containing module (ECM) of FIG. 1 and associated components;

FIG. 4 is a diagram showing an example primary drug containing modules (PDCM) of FIG. 1 and associated components;

FIG. 5 is an illustration of an example ECM;

FIG. 6 is an illustration of an example PDCM;

FIG. 7 is an illustration of an example integrated device with an ECM mated to a PDCM;

FIG. 8 is an illustration of an example ECM with a plurality of interconnect interfaces;

FIG. 9 an illustration of an example integrated device with an ECM with a plurality of interconnect interfaces mated to a plurality of PDCMs; and

FIGS. 10-13 are process flow diagrams of example methods for producing the drug dispensing ophthalmic device with integrated modular elements.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within at least a portion of a patient's eye for optometry or ophthalmology purposes (e.g., for diagnosis, surgery, vision correction, disorder treatment, or the like). An ophthalmic device may include integrated components related to (1) storage and delivery of one or more drugs and (2) active electronic components that can be used to control one or more functions of the ophthalmic device. The one or more integrated components may be encapsulated within at least a portion of the ophthalmic device. An example of an ophthalmic device that is worn on an exterior of an eye and that facilitates pharmaceutical release can be referred to as a “drug dispensing contact lens”, a “smart contact lens”, or any combination thereof.

As used herein, the term “module” refers to one of a set of independent self-contained units that can be used to construct a more complex structure. Modules can be passive (containing no power source and electronic components that do not require electricity to control an electrical signal) or active (containing active electronic components that use electricity to control an electrical signal and/or a power source for the electricity/electrical signal). Two or more of the modules can be connected and combined to form an integrated device, which may be encapsulated within a body of an ophthalmic device. As an example, modules can include one or more connectors or interfaces able to be combined with each other.

As use herein, the term “interconnect interface” refers to an electrical connection component included in each module. When two interconnect interfaces, each on a different module, are mated together, an electrical connection is formed such that each of the modules is in electrical communication. An interconnect interface can be male (e.g., a plug), female (e.g. a socket), or universal. A male interconnect interface can only be mated with a corresponding female interconnect interface and universal interconnect interface can mate with a similar universal interconnect interface. An interconnect interface can be active or passive. A material may be used to form and/or hold the interconnect interfaces together.

As used herein, the term “electronic control module” or “ECM” refers to an electronically active module that must include at least one active electronic component (and may include one or more passive electronic components). In some instances active and/or passive components of the electronic control module may be in the form of microelectronics. For example, an ECM can include electronic components for communication, power, and programming purposes, such as, but not limited to, an application specific integrated circuit (ASIC), an oscillator, a battery, a capacitor, and an antenna.

As used herein, the term “primary drug container module” or “PDCM” refers to an electronically passive module that includes components typically used for drug delivery, including at least a reservoir and the drug in the reservoir. The PDCM can include a single drug or a combination of drugs. It should be understood that multiple PDCMs, which can include different drugs or the same drug, can be combined with the ECM.

As used herein, the terms “electronically active,” “active,” and the like refer to a component that has/relies on an external power source to control or modify electrical signals. The ECM can be at least partially active (e.g., can include a battery power source or other type of power source).

As used herein, the terms “passive,” “electronically passive”, and the like refer to a component that does not rely on an external power source, but instead can communicate with an active component to receive controlled or modified electrical signals. The PDCM(s) are entirely passive and reliant on the ECM for power.

As used herein, the term “reservoir” refers to a storehouse for a drug (e.g., a volume or an amount of the drug) with a portion having an opening for release of the drug. The opening may be covered with an electrode or another substance to prevent release of the drug. In some instances, the covering can facilitate release of the drug from the reservoir. For example, at least a portion of the covering can be an electrode that can electrodissolve to facilitate the release of the drug in response to an electrical signal.

As used herein, the term “drug” refers to one or more substance (e.g., liquid, solid, or gas) used for the treatment, symptom relief, or palliative care of one or more maladies (e.g., a disease, disorder, injury, or the like) Examples of such maladies include, but are not limited to, dry eye, macular degeneration, glaucoma, retinopathy, etc. For example, the drug can be a pharmaceutical, saline solution, over the counter eye drops, or the like. The term “drug” can be used interchangeably with the terms “therapeutic” and “pharmaceutical”.

As used herein, the term “electrode” refers to a conductive solid (e.g., including one or more metals, one or more polymers, or the like) that receives/transmits an electrical signal. For example, an electrode covering a reservoir can be a thin film gold electrode within the passive PDCM and can receive a signal that activates electrodissolution from the active ECM. Other electrodes in the PDCM can receive a signal and/or power from the active ECM for other processes and purposes as is known in the art.

As used herein, the term “electrodissolution” refers to a process for dissolving a solute using an electrical catalyst. In one non-limiting example, application of an electrical signal to a solid metal can cause the solid metal to electrodissolve into separate molecules.

As used herein, the term “encapsulate” refers to fully enclosing an object (e.g., an integrated device) within something else (e.g., a body of an ophthalmic device).

As used herein, the terms “patient”, “subject”, “user”, and the like can be used interchangeably and can refer to an animal (e.g., a human) suffering from a condition that can be treated with a drug dispensing ophthalmic device.

### II. Overview

Actively controlled drug dispensing contact lenses and other actively controlled ophthalmic and implantable devices can be useful for directly releasing one or more drugs to a particular location of interest in or on a patient's body (e.g., directly to a surface of an eye). Such devices include at least passive and active electronic components and storehouses for the one or more drugs to be delivered. For example, in a drug dispensing ophthalmic device one or more drug reservoirs can be connected with electronic components to form a fully programmable devices for on-demand drug delivery that can optimize patient specific treatment regimens and simultaneously address patient compliance to improve outcomes in the treatment of chronic diseases/disorders (e.g., glaucoma, dry eye disease, and the like).

However, significant challenges arise in the packaging, storage, and sterilization of devices that include one or more drugs and active electronic components within a single device because instructions and requirements for each can often be counter to one another or even unnecessary in some cases. Thus, packaging, storage, and sterilization requirements for a full device must meet requirements suitable for all the active electronic components (e.g., one or more power sources) and each of the drugs used which can add to sterilization and storage related expenses and decrease the shelf life of the entire device to the lowest component or drug shelf life. For example, a given drug may require refrigeration and have a shelf life of 90 days, while a second drug may require room temperature storage for a shelf life of 180 days but have only a 60 day shelf life under refrigeration and electronic components could last for 1 year regardless of temperature, but a complete device would have to be stored under refrigeration and would only have a 60 day shelf life. Similar examples can be given for sterilization and packing requirements.

Accordingly, described herein are systems and methods for forming a drug dispensing ophthalmic device with integrated modular elements that include an active electronic control module (ECM) and one or more passive primary drug container modules (PDCM). The ECM can supply power and the PDCM can use the power. The modular elements can be implemented independently and, thus, able to be stored independently. Thus, drug packaging, sterilization, and storage requirements can be decoupled from one another and/or from packaging, sterilization, and storage requirements for electronic components until such time as each module is combined and encapsulated within a body of an ophthalmic device to create a drug dispensing ophthalmic device.

### III. System

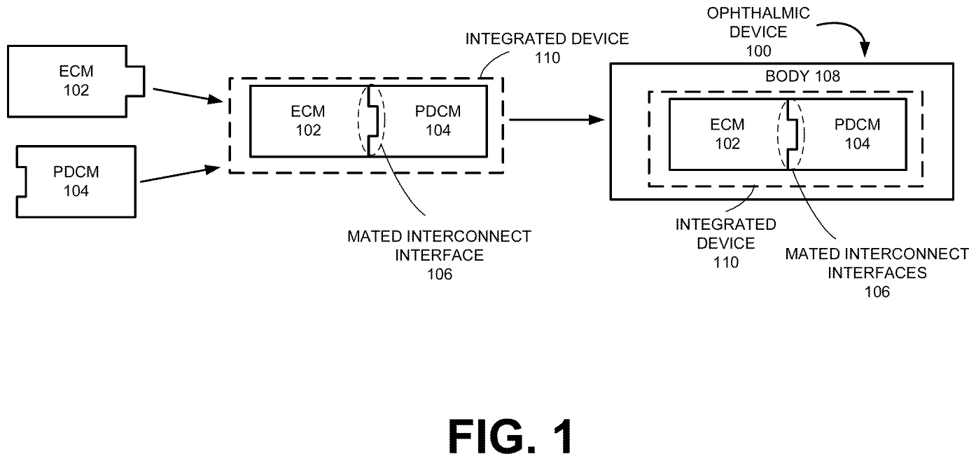

A drug dispensing ophthalmic device 100 (FIG. 1), such as a drug dispensing contact lens, can deliver one or more drugs to a patient's eye. The drug dispensing ophthalmic device can be used to treat disorders/conditions/symptoms of a patient's eye including, for example, dry eye, macular degeneration, glaucoma, retinopathy, and the like. The one or more drugs for treatment can be housed in one or more reservoirs whose release is electronically controlled. The usable life of the drug dispensing ophthalmic device may be limited due to differences in packaging, sterilization, and storage requirements of one or more drugs and the associated electronic components (which must be stored together in a traditional ophthalmic device). This creates significant challenges in the packaging, storage, and sterilization of the one or more drugs and the electronic components within a single device because the requirements for packing, sterilization, storage, and shelf life of the one or more drugs are often counter to the requirements of the electronic components and in many cases the requirements for the one or more drugs may be unnecessary for the integration and packaging of the electronic components.

These challenges can be avoided by splitting the portion of the device housing the one or more drugs and the associated active electronic component(s) into separate modules, creating a modular drug dispensing ophthalmic device 100. The separate modules can be sterilized, stored, and selected independently, enabling improved storage and sterilization options, interchangeable module combinations and providing for complete flexibility in the integration of one or more drug-containing modules (passive PDCM 104, described in detail below) with a single associated electronic component(s) module (active ECM 102, described in detail below). It should be noted that a plurality of drug-containing modules (each having a passive interconnect interface) can be mated to a single associated electronic component(s) module (having a plurality of interconnect interfaces), the combination(s) can be fixed or unfixed. Each module can be implemented independently to decouple the packaging, sterilization, and storage requirements of the different modules and enable the different modules to be combined at a later time for final implementation as a drug dispensing ophthalmic device 100. This modular approach enables a high level of flexibility in the implementation of customized combination-therapy ophthalmic devices 100 (e.g., smart contact lenses) with reduced regulatory and manufacturing costs realized through individualize production of the modules.

The active electronic component(s) can be housed in an electronic control module (ECM) 102. The one or more drugs can be stored in one or more primary drug container modules (PDCMs) (a single PDCM 104 is shown in FIG. 1). The ECM 102 can include one or more electronic components including active electronic components such as one or more power generators, while the PDCM can be entirely passive (e.g., including non-electronic components or passive electronic components). In other words, when connected the ECM 102 can supply power to the PDCM 104, while the PDCM 104 includes no active electronics, components, chips, semiconductor-based materials, or the like, but can include components that use the power generated by the ECM 102.

The ECM 102 and the PDCM 104 (or each of the one or more PDCM) can each include an interconnect interface that when mated, as a mated interconnect interface 106, can establish an electrical connection between the ECM 102 and the PDCM 104 (or each of the one or more PDCM) such that the ECM 102 provides power to the passive PDCM 104 (or each of the one or more PDCM). As shown in FIG. 1, the ECM and the PDCM 104 can each include at least one interconnect interface that can be shaped and/or otherwise formed to mate together (at mated interconnect interface 106) to form an integrated device 110. It should be understood that the shapes shown in FIG. 1 are for illustrative purposes only. The integrated device 110 can be encapsulated within a body 108 to form the complete drug dispensing ophthalmic device 100. The body 108 can be made, for example, of a soft, flexible, biocompatible material suitable/safe for optical wear, such as a polymeric material like polymethyl methacrylate (PMMA), polyhydroxyethylmethacrylate (polyHEMA), silicone hydrogel, silicon-based polymer(s) like fluoro-silicon acrylate, silicone elastomer, combinations thereof, or the like.

Within the integrated device 110, the active components of the ECM can provide power to the passive components of the PDCM 104 via the electrical connection formed by the mated interconnect interface 106. Thus, prior to integrating the integrated device 110 drug packaging, sterilization, and storage requirements (of the PDCM 104) can be decoupled from one another and/or from packaging, sterilization, and storage requirements for electronic components (of ECM 102). The decoupled modules can be kept separate until such time as the integrated device 110 is formed and encapsulated within the body 108 to create the drug dispensing ophthalmic device 100.

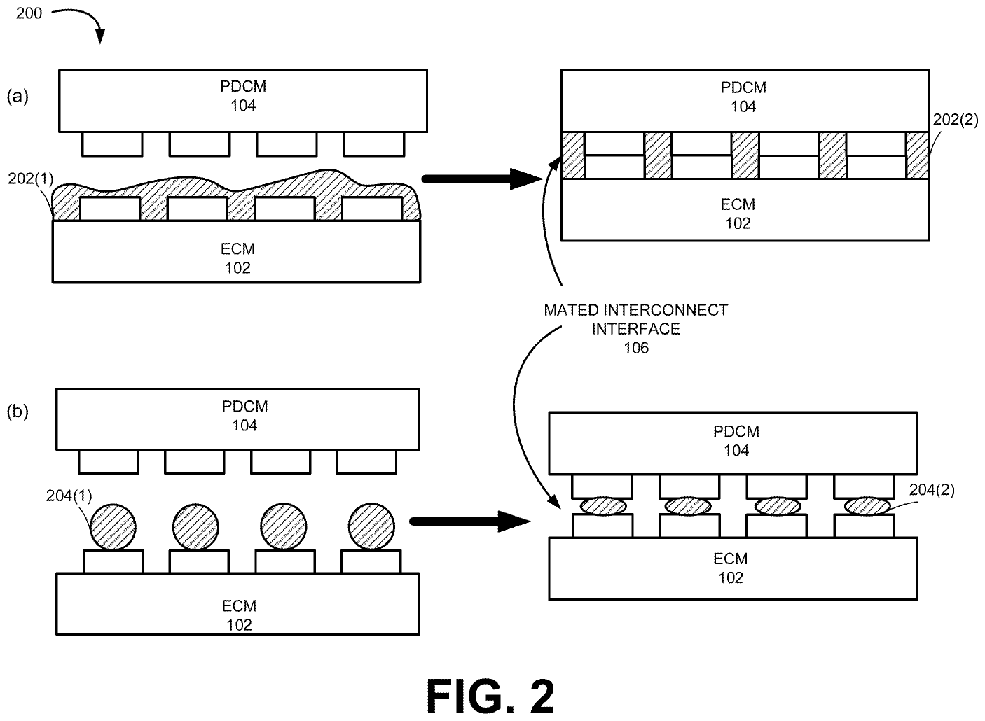

FIG. 2 shows two different non-limiting example illustrations 200(a) and 200(b) for establishing an electrical connection between an interconnect interface of the ECM 102 and the passive interconnect interface of the PDCM 104. In both 200(a) and (b), an electrically conductive adhesive (shown as 202(1) and 202(2) in example (a) and 204(1) and 204(2) in example (b)) can be used to attach the active interconnect interface of the ECM 102 to the passive interconnect interface of the PDCM 104 so that the power provided by the ECM 102 can be conducted through to the PDCM 104. The electrically conductive adhesive can include electrically conductive particles (e.g., silver, nickel, copper, graphite, or the like) suspended in a sticky component (e.g., a varnish, a synthetic resin, silicon, acrylate, epoxy, or the like) that holds the electrically conductive particles together. In some instances, the electrically conductive particles can make up the majority of the total mass of the electrically conductive adhesive.

In example (a), an epoxy based process is shown where a surface of the interconnect interface of the ECM 102 can be covered by a layer of malleable and/or paste like electrically conductive adhesive 202(1) that the interconnect interface of the PDCM 104 can be pressed into to form the mated interconnect interface 106. The malleable and/or paste like electronically conductive adhesive 202(1) can fill and seal any gaps in the mated interconnect interface 106 and can hardened (e.g., by application of time, heat, or the like) to become the binding electronically conductive adhesive 202(2). In example (b), a reflow based process is shown where an electrically conductive adhesive 204(1) can be present in small amounts on at least one portion of a surface of the interconnect interface of the ECM 102 and can be compressed to cover at least a portion of a surface of the interconnect interface of the PDCM 104, shown as electrically conductive adhesive 204(2). It should be understood that interconnect interfaces of ECM 102 and PDCM 104 can be shaped/configured in any manner (beyond the shapes illustrated in FIGS. 1 and 2) to facilitate the electrical connection between the active ECM 102 and the passive PDCM 104. Additionally, the mated interconnect interface 106 between ECM 102 and the PDCM 104 can be formed by one of any number of mechanisms/processes beyond those illustrated as examples in FIG. 2, including, thermocompressive bonding, flip-chip bonding, asymmetric conductive pastes, laser welding, reflow, or the like. The integrated device 110, as illustrated in FIG. 1, can be formed upon the mating the interconnect interfaces of the ECM 102 and the PDCM 104. The integrated device 110 can then be encapsulated within the body 108 to form the ophthalmic device 100 for sale and/or use.

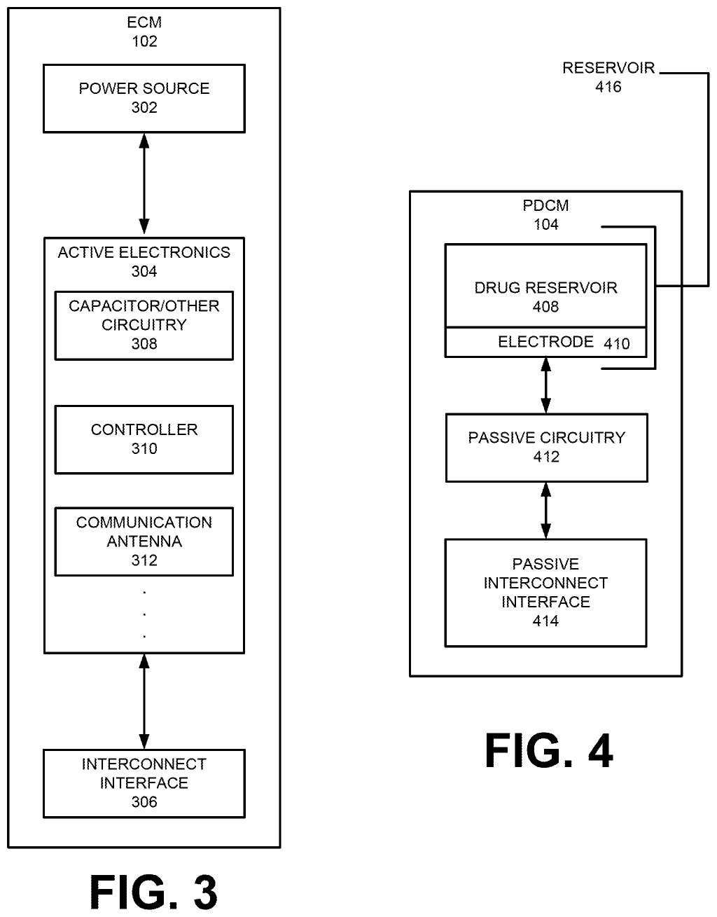

FIG. 3 shows a diagram of the main components of the ECM 102. The ECM 102 can be electrically active, having at least one component configured to generate/supply power as an electrical energy source (in other words, one or more circuit components that are entirely responsible for the flow of current). As illustrated, the component configured to generate/supply power as the energy source within the ECM 102 can be power source 302 (which may include one or more voltage sources, one or more current sources, one or more batteries, one or more links to an external power source, one or more semiconductor devices (e.g., transistors, etc.), and/or the like). The ECM 102 can also include electronic components (active electronics 304) that are not power generating, but use the power, including a capacitor/other circuitry 308, a controller 310 (e.g., an application specific integrated circuit (ASIC)), an antenna (e.g., a communication antenna 312), an oscillator, a battery, or the like. The ECM 102 can also include at least one interconnect interface (a single interconnect interface 306 is shown in FIG. 3, but more than one can be included in an ECM). At least a portion of the ECM 102 can be embodied on at least one substrate. The at least one substrate can be one or more polymer substrates. In some instances, at least a portion of the at least one substrate can be biocompatible

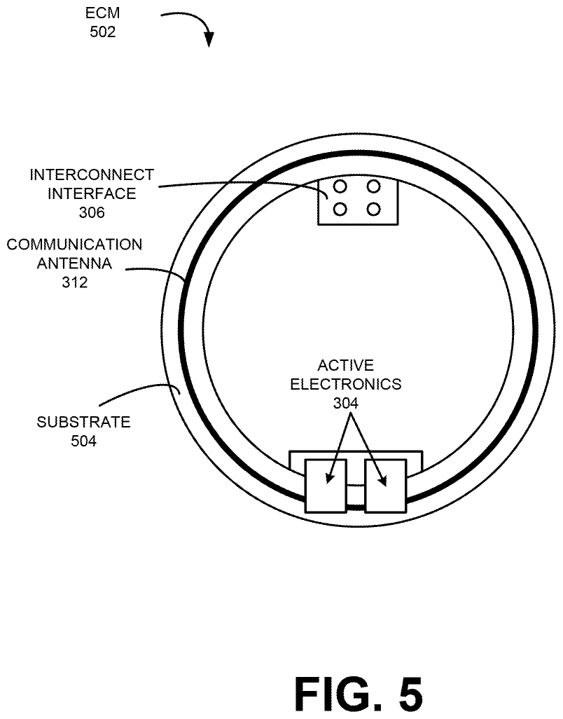

FIG. 4 shows a diagram of the main components of the PDCM 104. The PDCM 104 can be electrically passive having at least one component that can use electrical energy but no components that can generate electrical energy. The PDCM 104 can include non-electronic components and passive electronic components (in other words, components that are not active and cannot generate power) disposed on another substrate. The non-electronic components can include one or more reservoirs 416 that can include a drug reservoir 408 (also referred to as a discrete storage unit) that can hold a volume of at least one drug (not shown in FIG. 4) and can be covered by electrode 410 that is able to dissolve or otherwise open when exposed to an electrical signal (e.g., an electrical signal generated by a component of the ECM 102 when the integrated device 110 is formed). The PDCM 104 can also include passive circuitry 412 of any kinds that cannot generate an electrical signal but may use and/or communicate the electrical signal. The PDCM 104 can also include a passive interconnect interface 414 (passive because can only receive an electrical signal). At least a portion of the PDCM 104 can be embodied on at least one other substrate (separate from the at least one substrate of the ECM). The at least one other substrate can be one or more polymer substrates. In some instances, at least a portion of the at least one other substrate can be biocompatible.

As noted, the ECM 102 can include at least one interconnect interface (a single interconnect interface 306 is shown in FIG. 3) and the PDCM 104 can include a passive interconnect interface 414 (as shown in FIG. 4). Each PDCM can include a single passive interconnect interface, but a single ECM 102 can include one or more interconnect interfaces depending on the number of PDCMs the ECM 102 may be mated to. Using the example with the ECM 102 with one interconnect interface 306, the interconnect interface 306 can be configured to mate (e.g., in ways described above) to the passive interconnect interface 414 of the PDCM 104. The interconnect interface 306 and the passive interconnect interface 414 can be of any size/shape/configuration to facilitate mating therebetween. As noted with respect to FIG. 2, the mating can include an electrically conductive adhesive to establish the electrical connection between the active ECM 102 and the passive PDCM 104. When the interconnect interface 306 is mated to the passive interconnect interface 414, power generated by the ECM 102 can be used by components of the ECM 102 and components of the passive PDCM 104. It should be noted that the ECM 102 and the PDCM 104 can be implemented independently and, thus, can be stored independently before mating.

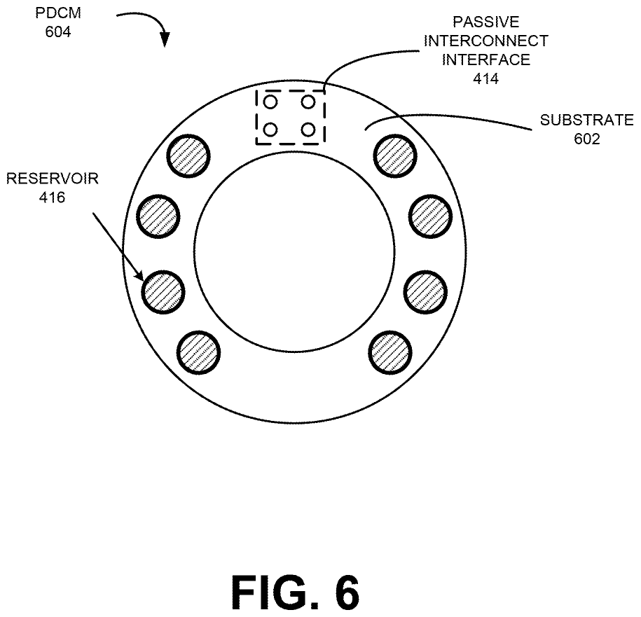

FIG. 5 is an illustration of an example ECM 502 with a non-exclusive shape and component layout. Although not meant to be limiting, the ECM 502 is shown shaped like ring and including embedded electronics represented as active electronics 304 (including an application specific integrated circuit (ASIC), oscillator, battery and capacitor) and communication antenna 312, which may be for communication and programming purposes. These embedded electronics can be disposed at least partially on and/or in a substrate 504 (or one or more substrates) in any manner. The substrate can be, for example, PMMA, cyclic olefin polymers (COP/COC) Parylene, PET, polyurethane, polyimide, rigid gas permeable fluorosilicone acrylate, liquid crystal polymer, silicon-based polymers, silicone acrylate, and the like.

The ECM 502 can also include at least one interconnect interface (shown as interconnect interface 306), each of which can be mated with a passive interconnect interface of a PDCM to form an electrical connection and connect the ECM and PCDM. One interconnect interface 306 is shown for ECM 502, but any number can be included in the ECM in any position relative to the substrate 504. The ECM 502 can be sterilized using electronic-safe techniques (e.g., ETO, autoclave, or the like) including techniques that cannot be used on components that include one or more drugs (e.g., for health and safety reasons, would destroy the drug, or the like). The ECM 502 can then be shipped and/or stored (e.g., in electronic discharge safe packaging) until the ECM 502 is selected for use under the desired sterile conditions.

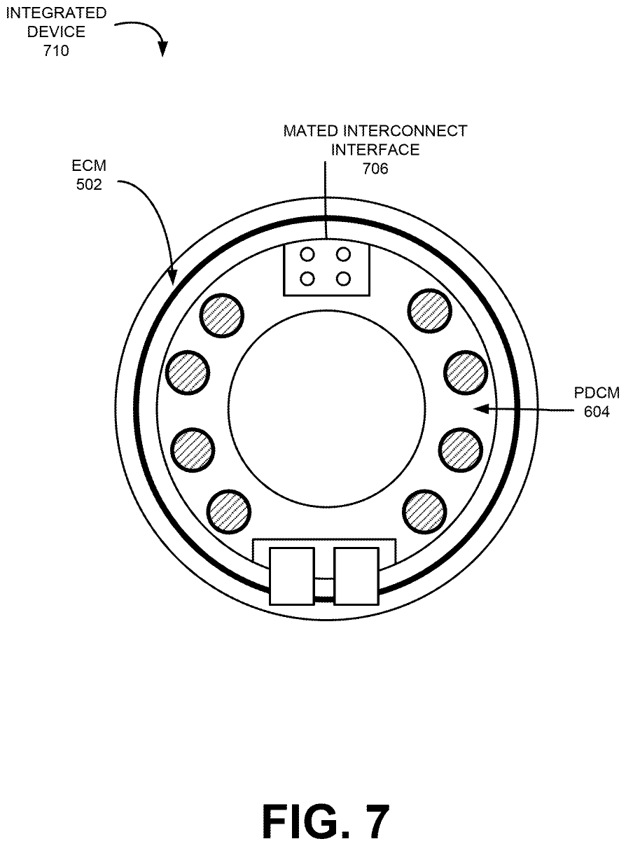

FIG. 6 is an illustration of an example PDCM 604 with a non-exclusive shape and component layout. The PDCM 604 is shown shaped like a ring and can include at least one reservoir 416, passive circuitry (not shown), and a passive interconnect interface 414 at least partially on and/or in a substrate 602. The passive circuitry can for example, connect the electrodes covering the reservoirs 416 to the passive interconnect interface 414. The PDCM 604 can include a number (e.g., at least one, but only limited by the size of the reservoir(s) and/or the size of the PDCM 604) of reservoirs 416, which are discrete storage units that may be of equal or varying dimensions and can range in volume from 1 pL-100 nL. For example, one PDCM 604 can include several hundred storage units (each containing the same drug, different drugs and/or combinations of one or more drugs). Additionally, each reservoir 416 can be uniquely addressable via an electronic lead (e.g., passive circuitry) that can connect at least one portion of a discrete drug storage unit (e.g., an electrode covering the reservoir opening) and terminate at the other end at the passive interconnect interface 414. The passive interconnect interface 414 (and the PDCM 604 as a whole) can be completely passive until mated with the corresponding interconnect interface on the ECM, which allows the PDCM 604 to accept electronic control input (e.g., electrical signals) from the ECM. It should be noted that the PDCM 604 can contain no active electronics, components, chips or semiconductor-based materials. The metal film (e.g., electrode) covering each discrete storage unit (e.g., drug reservoir) can enable the at least one drug stored in each of the discrete storage units to be programmatically released on-demand through an electrodissolution process (where the at least one drug is released when the metal film is dissolved), for example. Alternative embodiments may include, for example, selective electrothermal degradation through local joule heating.

The PDCM 604 can be sterilized using techniques compatible with industry standard primary drug storage containers (e.g., gamma radiation, ETO, autoclave, e-beam, or the like) prior to at least one drug being dispensed into the drug reservoir(s). Pre-washing or other preparations (e.g., approved hydrophobic or type-1 glass coatings) can also and/or alternatively be conducted prior to at least one drug being dispensed into the drug reservoir(s). The at least one drug can then be dispensed into the drug reservoir(s) via ink-jet printing, micro-injection, micro dispensing, syringe dispensing, or other method capable of pL-nL volume control. Lyophilization (freeze-drying) of the final deposit can also be performed to remove residual moisture.

The PDCM 604 can have with a final sealing step or capping step (e.g., coating, bonding, welding, etc.) of adding the covering (e.g., thin metal electrode) over the drug reservoir(s) to create a reservoir(s) 416 (otherwise called discrete storage unit(s)) to keep the at least one drug under hermetic or near hermetic storage conditions, which can be implemented in-line or as part of standard lyophilization process. The PDCM 604 can then be packaged in sterile packaging and stored under recommended conditions (e.g., freezer, dry box, dark room, shelf, or the like) to promote or prolong drug stability and shelf-life. The PDCM 604 can be stored in this stable condition ready for shipping and/or until the module is selected for use and implementation in an ophthalmic device. The at least one drug can also be formatted for “dry” storage (e.g., as a solid rather than as a liquid) in the reservoir(s) 416. Due to the nature of the “dry” storage, conventional degradation risks typically encountered in bottled pharmaceuticals (e.g., decomposition, pH shift, solubility limits, stability, or the like) are mitigated further extending storage life in the PDCM 604.

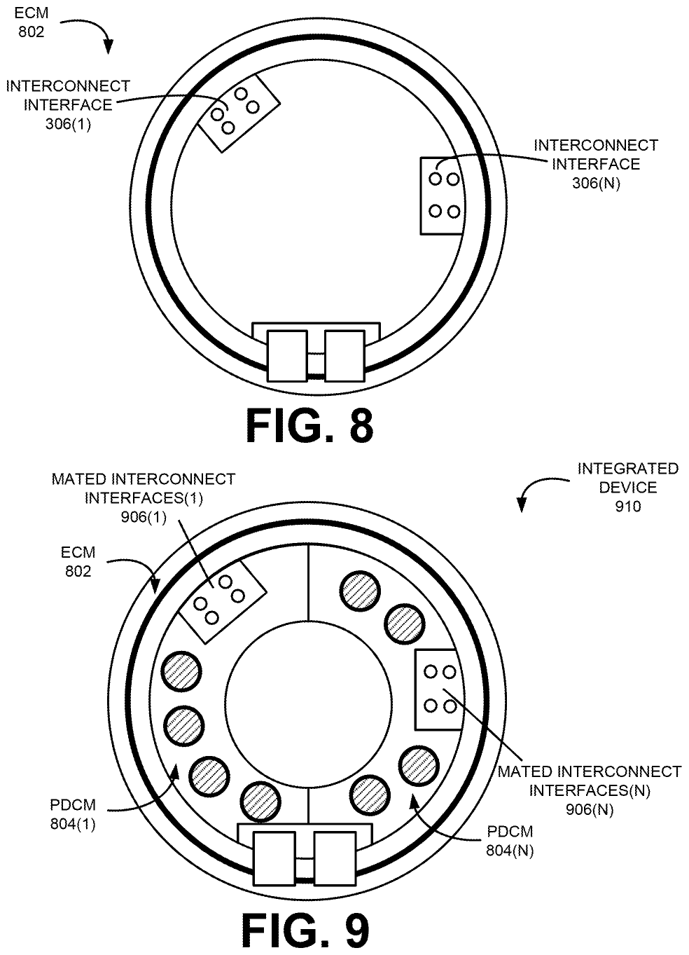

FIG. 7 is an illustration of an example integrated device 710 with an ECM 502 mated to a PDCM 604 at a mated interconnect interface 706 (e.g., as described with respect to FIG. 2). This modular approach employing the ECM 502 and the PDCM 604 can enable a high level of flexibility in the implementation of customized drug delivery (even combination therapy) ophthalmic devices (e.g., smart contact lenses). The combined ECM 502 and PDCM 604 can form the integrated device 710 that can be a complete electronic delivery system for at least one drug. An electrical connection can be established between the interconnect interface of the ECM 502 and the passive interconnect interface of the PDCM 604 at the mated interconnect interface 706. The interconnect interface of the ECM 502 and the PDCM 604 can be mated by one of any number of mechanisms/processes, including thermocompressive bonding, flip-chip bonding, asymmetric conductive pastes, laser welding, reflow, or the like. The integrated device 710 can be encapsulated in body of an ophthalmic device including a soft flexible biocompatible material suitable for ocular wear, such as polymeric material like polymethylmethacrylate (“PMMA”), polyhydroxyethylmethacrylate (“polyHEMA”), silicone hydrogel, silicon based polymers (e.g., flouro-silicon acrylate), silicone elastomer, or combinations thereof. The full ophthalmic device can then be used to deliver the at least one drug to treat a disorder, illness, and/or symptom of the eye.

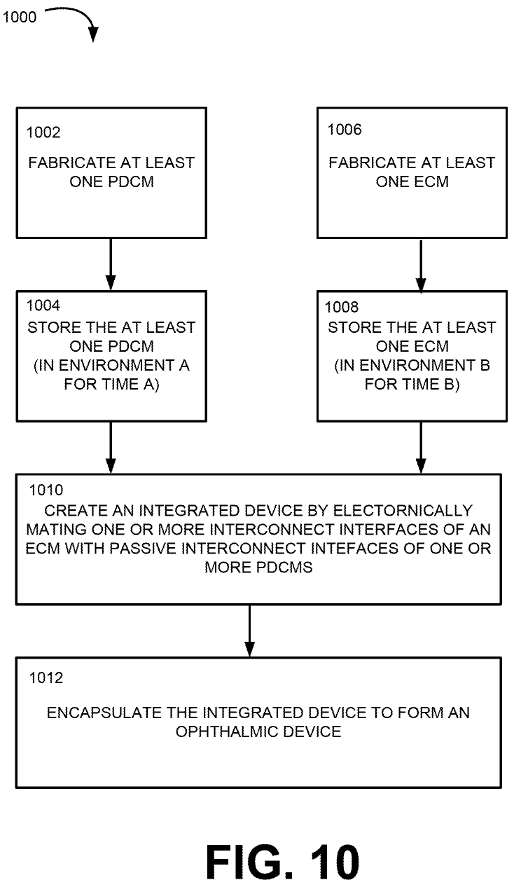

FIG. 8 is an illustration of an example ECM 802 with a plurality of interconnect interfaces 306(1)-306(N). Each interconnect interface 306(1)-306(N) can connect to a single PDCM (so that once ECM 802 can power multiple PDCMs). Each of the plurality of interconnect interfaces 306(1)-(N) can mate to a single passive interconnect interface of a passive PDCM (e.g., as described with regard to FIG. 2). Each of the plurality of interconnect interfaces 306(1)-(N) can be positioned in any manner on and/or relative to any other portion of the ECM 802 to facilitate connection with a passive interconnect interface of one or more PDCM. In one example, each of the plurality of interconnect interfaces 306(1)-(N) may be movable, such as along the curve of the substrate of the ECM to facilitate connection of differently shaped PDCMs.

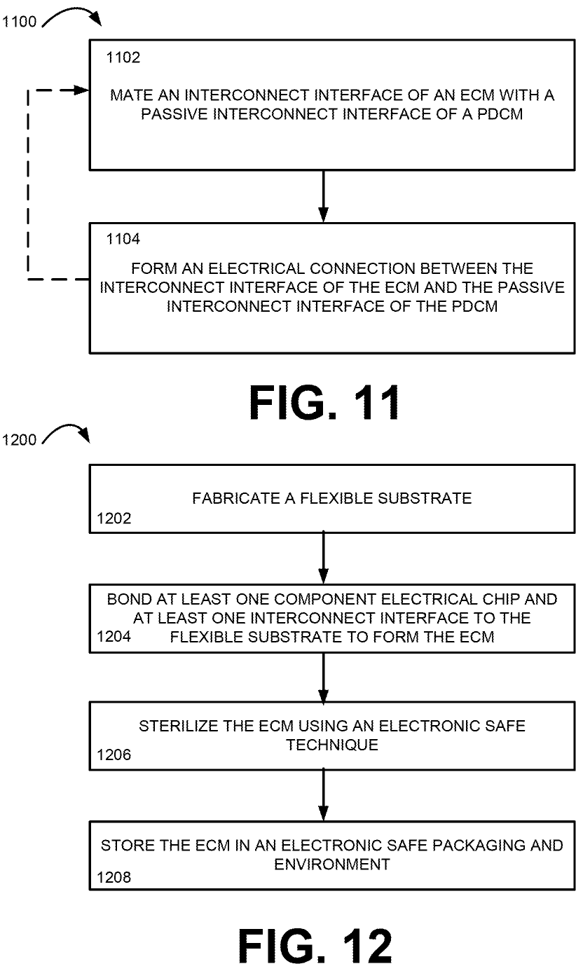

FIG. 9 an illustration of an example integrated device 910 (that can be encapsulated within a body of an ophthalmic device) with an ECM 802 mated to a plurality of PDCMs 804(1)-(N). The example is intended to be non-limiting. Each mated interconnect interface (1)-(N) 906(1)-(N) can be formed by an interconnect interface of the ECM 802 and a passive interconnect interface of one PCDM 804(1)-(N). Each of the PDCMs 804(1)-(N) can include one or more reservoirs providing a stand-alone, sealed, and sterile storage environment for one or more drugs. In some instances, a drug or combination of drugs can be stored independently in each reservoir. In other instances, different drugs or combinations of drugs can be stored independently in each reservoir. Each PDCM 804(1)-(N) can, for example, include one drug or more than one drug having similar storage conditions so each PDCM can be stored for maximum shelf life before being combined into integrated device 910. Additionally, each PDCM 804(1)-(N) can be chosen to form the device 910 for the greatest amount of customization (e.g., based on drug type(s), prescribed combination therapies, volumes of drug(s), length of wear time and/or lifetime of the ophthalmic device as a whole, or the like). Each drug filled reservoir can have an opening covered with an electrode. Each electrode can be configured to receive a signal from the ECM 802 when it is a time to release the drug and upon receiving the signal from the ECM 802 can open the respective reservoir (e.g., by electrodissolution or other process that dissolves or otherwise opens the electrode). Each of the plurality of reservoirs can be uniquely addressable via an electronic lead formed by the mating of the passive interconnect interface of the PDCM 804(1)-(N) and an interconnect interface (one of the plurality of interconnect interfaces) of the ECM 802.

### IV. Methods

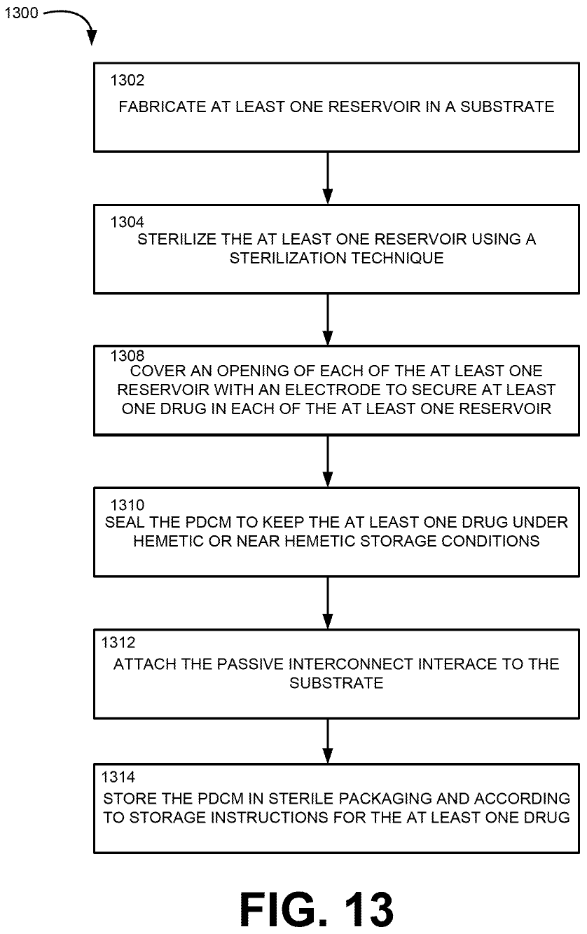

Another aspect of the present disclosure can include example methods 1000, 1100, 1200, and 1300 (shown in FIGS. 10, 11, 12, and 13) for producing the drug dispensing ophthalmic device (e.g., a drug dispensing contact lens) with integrated modular elements (examples of which are shown as components of the ophthalmic device 100 in FIG. 1). The methods 1000, 1100, 1200, and 1300 are illustrated as process flow diagrams with flowchart illustrations that can be implemented by/with the ECM and PDCM modules shown in FIGS. 1-9.

For purposes of simplicity, the methods 1000, 1100, 1200, and 1300 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 1000, 1100, 1200, and 1300. It should be noted that one or more steps of the methods 1000, 1100, 1200, and 1300 can be executed by a hardware processor.

Referring now to FIG. 10, illustrated is a method 1000 for forming a drug dispensing ophthalmic device (e.g., drug dispensing ophthalmic device 100). The drug dispensing ophthalmic device can include an electronic control module (ECM) mated a primary drug container module (PDCM). It should be noted that a single ECM can accept one PDCM (e.g., ECM 502 mated to PDCM 604 in FIG. 7), but the single ECM (shown, for example, in FIG. 8) can also accept multiple PDCMs (shown, for example, in FIG. 9). Each PDCM can have a single passive interconnect interface for mating to one interconnect interface of an ECM, but each ECM can have one or more interconnect interfaces. This modular interchangeability enables a single platform to provide monotherapy and/or a fixed or unfixed combination of therapeutics through straightforward selection of PDCMs without significant process deviations. More generally, the modularity enables customized combination-therapy delivery profiles which is currently impossible or impractical with topical administration.

At 1002, the at least one PDCM can be fabricated (e.g., according to the method 1300 of FIG. 13). After fabrication, at 1004, the at least one PDCM can be stored in an environment (environment A) for a time (time A). The at least one PDCM can be fabricated and stored so as to satisfy packaging, sterilization, and storage requirements of one or more drugs. For example, the PDCM can be stored in a way favorable for one or more drugs therein, such as in the dark, under refrigeration, under freezer temperatures, or the like. The one or more drugs packaged in the PDCM can be stored as “dry” (e.g., as a solid rather than suspended in a liquid). Due to the nature of the “dry” storage, conventional degradation risks typically encountered in bottled pharmaceuticals (e.g., decomposition, pH shift, solubility limits, stability, etc.) are mitigated further extending storage life in the PDCM. The one or more PDCM can be entirely passive (e.g., includes no active electronics, components, chips, semiconductor-based materials, or the like). It should be noted that different PDCMs with different drugs can be stored in different environments/conditions from one another.

At 1006, at least one ECM can be fabricated (e.g., according to the method 1200 of FIG. 12). After fabrication, the at least one ECM can be stored in another environment (e.g., environment B) for another time (time B) at 1008. The at least one ECM can be fabricated and stored so as to satisfy packaging, sterilization, and storage requirements for electronic components (and these requirements may be different from/contrary to those of the drug(s) of the one or more PDCM). The ECM can house active and passive electronic components, including a power generator (an active component) so that the ECM is considered electronically active (in other words, the ECM can supply power to the one or more PDCM when mated together).

An ECM can be selected from storage along with one or more PDCM (selected from the different storage environment) based on the custom or semi-custom treatment prescribed for a patient (e.g., for a single patient or a similar group of patients). For example, at least two drugs can be chosen that interact for improved efficacy of treatment of the disorder of the eye (one PDCM may house each of the at least two drugs, or two or more PDCM may each house on of the at least two drugs). At 1010, an integrated device can be created by electronically mating the one or more interconnect interfaces of an ECM with interconnect interfaces of one or more PDCM (described in more detail with respect to FIG. 11). The mating can form an electrical connection between one or more interconnect interfaces of the ECM each to a passive interconnect interface of the one or more PDCM and allow an electrical signal to be transferred from the ECM to each PDCM. At 1012, the integrated device (e.g., integrated device 110) can be encapsulated within a body of an ophthalmic device (e.g., ophthalmic device 100). The ophthalmic device can be used to treat the disorder of the eye, such that at least one drug is actively dispensed to the eye using the ECM from a PDCM. In one example the at least one drug can be actively dispensed to the eye from a first PDCM and at least one other drug can be dispensed to the eye using the ECM and the at least a second PDCM. As an example, the treatment of the disorder of the eye can include a predetermined schedule of delivery of the at least one drug and/or the at least one other drug to the eye. As another example, the treatment of the disorder of the eye can be based on feedback from one or more sensors associated with the patient (e.g., measuring one or more physiological signals associated with a change in a state of the disorder being treated and/or by manual choice of the user.

Referring now to FIG. 11, illustrated is a method 1100 for creating one or more mated interconnect interfaces between an ECM and one or more PDCMs. At 1102, one interconnect interface of an ECM can mate with a passive interconnect interface of a PDCM. For example, the mating can be done as shown and described with respect to FIG. 2. The mating can be epoxy boding, thermocompressive bonding, flip-chip bonding, asymmetric conductive pastes, laser welding, reflow, or the like. At 1104, an electrical connection can be formed between the interconnect interface of the ECM and the passive interconnect interface of the PDCM. This process can be repeated for each additional PDCM chosen for the ophthalmic device to treat the certain disorder of the eye.

It should be noted that a plurality of drug containing PDCMs can be mated to different interfaces on a single associated ECM and the combination(s) of the PDCMs can be fixed or unfixed and each PDCM can include a single drug and/or combinations of one or more drugs. Each module can be implemented independently to decouple the packaging and storage requirements of the different modules and enable the different modules to be combined at a later time for final implementation as a drug dispensing ophthalmic device. This modular approach enables a high level of flexibility in the implementation of customized combination-therapy ophthalmic devices with reduced regulatory and manufacturing costs realized through individualize production of the modules.

Referring now to FIG. 12, illustrated is a method 1200 for forming an ECM (e.g., this method can be used to fabricate the ECM of FIGS. 3, 5 and/or 8). At 1202, a flexible substrate can be fabricated. The flexible substrate can be shaped for encasement in an ophthalmic device and connection with one or more PDCMS, for example, the ophthalmic device can be a drug dispensing contact lens and the substrate can be formed as a ring with a hole sized at least large enough for a patient to see through. At 1204, at least one component electrical chip (with a power source) and at least one interconnect interface can be bonded to the flexible substrate to form the ECM. Other required electronic components can be bonded to the flexible substrate as well. The bonding can be by any process known in the art. At 1206, the ECM can be sterilized using an electronic safe technique (e.g., ETO, autoclave, or the like). At 1208, the ECM can be stored in an electronic safe packaging and environment until needed for fabrication into an integrated device and then an ophthalmic device.

Referring now to FIG. 13, illustrated is a method 1300 for forming a PDCM (e.g., this method can be used to fabricate the PDCM of FIGS. 4 and/or 6). At 1302, at least one reservoir can be fabricated in a substrate. The substrate can be fabricated in any shape and/or material safe for use in an ophthalmic device and integration with an ECM. For example, the substrate can be ring shaped and can be concentric with the ECM shape. The substrate can be shaped to include at least one drug reservoir and can be cleaned/sterilized according to standards for use with the body. Each of the at least one drug reservoirs can have a certain storage volume (so a known amount of the drug can be delivered). At 1304, the at least one reservoir can be sterilized using at sterilization technique approved for medical use (e.g., at least one of gamma radiation, ETO, autoclave or e-beam). The storage volume of each of the at least one reservoir can then be filled with at least one drug (e.g., via ink-jet printing, micro-injection, micro dispensing, syringe dispensing, or any other method capable of pL-nL volume control). At 1308, an opening of each of the at least one reservoir can be covered with an electrode (e.g., a thin metal film) to secure the at least one drug in each of the at least one reservoir. At 1310, the PDCM can be sealed to keep the at least one drug under hermetic or near hermetic storage conditions. This can be repeated (or done concurrently) for each PDCM. At 1312, the passive interconnect interface can be attached to the substrate. Passive circuitry connecting the passive interconnect interface and the electrode of the at least one reservoir can also be attached. At 1314, the PDCM can be stored in sterile packaging and according to storage instructions for the at least one drug.

From the above description, those skilled in the art will perceive improvements, changes and modifications. Such improvements, changes and modifications are within the skill of one in the art and are intended to be covered by the appended claims.

What is claimed is:

1. A method comprising:
creating an integrated device comprising:
mating an interconnect interface of an electronic control module (ECM) to a passive interconnect interface of a primary drug container module (PDCM);
forming an electrical connection between the interconnect interface of the ECM to the passive interconnect interface of the PDCM, wherein the ECM is active and the PDCM is passive; and
encapsulating the integrated device within a body of an ophthalmic device.

2. The method of claim 1, further comprising manufacturing and storing the ECM and the PDCM in different environments.

3. The method of claim 1, further comprising manufacturing the ECM, comprising:
fabricating a flexible substrate;
bonding at least one component electrical chip and at least one interconnect interface to the flexible substrate to form the ECM;
sterilizing the ECM using an electronic-safe technique; and
storing the ECM in an electronic safe packaging and environment.

4. The method of claim 1, further comprising manufacturing the PDCM, comprising:
fabricating at least one reservoir in a substrate, wherein each of the at least one reservoir comprises a storage volume;
sterilizing the at least one reservoir using at least one of gamma radiation, ETO, autoclave or e-beam;
filling the storage volume of each of the at least one reservoir with at least one drug;
covering an opening of each of the at least one reservoir with an electrode to secure the at least one drug in each of the at least one reservoir;
sealing the PDCM to keep the at least one drug under hermetic or near hermetic storage conditions;
attaching the passive interconnect interface; and
storing the PDCM in sterile packaging and according to storage instructions for the at least one drug.

5. The method of claim 1, further comprising:
mating another interconnect interface of the ECM with a passive interconnect interface of another PDCM; and
forming an electrical connection between the other interconnect interface of the ECM to the passive interconnect interface of the other PDCM.

6. The method of claim 5, further comprising storing the other PDCM separately from the PDCM based on different storage instructions for at least one drug in the PDCM and at least one other drug in the other PDCM.

7. An ophthalmic device comprising:
an electronic control module (ECM) comprising an interconnect interface;
a primary drug container module (PDCM) comprising a passive interconnect interface, wherein the passive interconnect interface of the PDCM is mated with the interconnect interface of the ECM, wherein the PDCM is passive and the ECM is active; and
a body comprising a biocompatible material safe for ocular wear.

8. The ophthalmic device of claim 7, wherein the ECM comprises active electronics of the ophthalmic device.

9. The ophthalmic device of claim 7, wherein the PDCM comprises no active electronics, components, chips, and/or semiconductor-based materials.

10. The ophthalmic device of claim 7, wherein the PDCM comprises at least one reservoir configured to store at least one drug and an electrode covering an opening of each of the at least one reservoir, wherein the electrode is configured to electrodissolve to release the at least one drug when a signal is delivered from the ECM.

11. The ophthalmic device of claim 7, wherein the PDCM further comprises a plurality of reservoirs configured to store one or more drugs.

12. The ophthalmic device of claim 11, wherein the one or more drugs are the same at least one drug per reservoir or different at least one drug per reservoir.

13. The ophthalmic device of claim 11, wherein each of the plurality of reservoirs is uniquely addressable via an electronic lead formed by the mating of the passive interconnect interface of the PDCM and the interconnect interface of the ECM.

14. The ophthalmic device of claim 7, wherein the ECM comprises electronic components disposed on a polymer substrate, where the electronic components comprise an application specific integrated circuit (ASIC), an oscillator, a battery, a capacitor, and/or an antenna.

15. The ophthalmic device of claim 7, wherein the ECM comprises a plurality of interconnect interfaces, further comprising:
at least one other PDCM comprising another passive interconnect interface,
wherein the other passive interconnect interface of the at least one other PDCM is mated with another of the plurality of interconnect interfaces of the ECM.

16. The ophthalmic device of claim 15, wherein each of the PDCM and the other PDCM comprise one or more reservoirs providing a stand-alone, sealed, and sterile storage environment for one or more drugs.

17. A method comprising:
choosing a first primary drug container module (PDCM) for a patient based on a disorder of an eye of the patient and at least one drug stored in the first PDCM;
choosing at least a second PDCM for the patient based on the disorder of the eye of the patient and at least one other drug stored in the second PDCM;
mating the first PDCM to a first interconnect interface of an electronic control module (ECM) to form an integrated device,
mating the at least the second PDCM to at least a second interconnect interface of the ECM to add to the integrated device; and
encapsulating the integrated device within in a body of an ophthalmic device.

18. The method of claim 17, wherein the choosing the first PDCM and the at least the second PDCM further comprises choosing at least two drugs that interact for improved efficacy of treatment of the disorder of the eye.

19. The method of claim 17, further comprising treating the disorder of the eye with the ophthalmic device, wherein the at least one drug is actively dispensed to the eye using the ECM and the first PDCM and the at least one other drug is dispensed to the eye using the ECM and the at least the second PDCM.

20. The method of claim 19, wherein treating the disorder of the eye with the ophthalmic device further comprises choosing a schedule of delivery of the at least one drug and the at least one other drug.
